(19) United States
(12) Patent Application Publication
(10) Pub. No.: US 2026/0109934 A1
(43) Pub. Date: Apr. 23, 2026

(54) ONLINE PURIFICATION AND SAMPLING APPARATUS, ONLINE VISUALIZED AUTOMATIC ENRICHMENT APPARATUS, ONLINE PURIFICATION AND SAMPLING AND VISUALIZED AUTOMATIC ENRICHMENT APPARATUS AND METHOD FOR DEEP-SEA MICROORGANISMS

(71) Applicant: GUANGDONG LABORATORY OF SOUTHERN OCEAN SCIENCE AND ENGINEERING (GUANGZHOU), Guangdong (CN)

(72) Inventors: Jingchun FENG, Guangdong (CN); Jianzhen LIANG, Guangdong (CN); Si ZHANG, Guangdong (CN); Yongji HUANG, Guangdong (CN); Guang YANG, Guangdong (CN)

(73) Assignee: GUANGDONG LABORATORY OF SOUTHERN OCEAN SCIENCE AND ENGINEERING (GUANGZHOU), Guangdong (CN)

(21) Appl. No.: 19/365,219

(22) Filed: Oct. 22, 2025

(30) Foreign Application Priority Data
Oct. 22, 2024 (CN) 202411477983.8

(51) Int. Cl. C12M 1/36; C12M 1/00; C12M 1/33; C12M 1/34

(52) U.S. Cl. CPC C12M 41/48; C12M 29/14; C12M 39/00; C12M 41/06; C12M 41/12; C12M 41/26; C12M 41/32; C12M 41/34; C12M 41/36; C12M 41/40; C12M 45/02

(57) ABSTRACT

The present invention provides an online purification and sampling and visualized automatic enrichment apparatus and method for deep-sea microorganisms. Through online sample pretreatment during microorganism sampling, automatic biological concentration and purification, online monitoring of environmental indicators during biological enrichment, online observation of biomass, and automatic enrichment and transfer of high-purity enriched materials, the present invention overcomes the shortcomings of the prior art, such as difficulties in purifying deep-sea microorganism samples under high-pressure environments, complex manual operations for enrichment and observation, low timeliness of biomass observation, and difficulties in real-time cell counting of the enriched materials. This effectively increases the enrichment rate and acquisition efficiency of difficult-to-culture deep-sea microorganisms, providing a fundamental way for enhancing deep-sea biological research and bioresource development efficiency.

### CROSS-REFERENCE TO RELATED APPLICATION

This application claims the priority benefit of China application serial no. 202411477983.8, filed on Oct. 22, 2024. The entirety of the above-mentioned patent application is hereby incorporated by reference herein and made a part of this specification.

### TECHNICAL FIELD

The present invention relates to the technical field of purification, enrichment, and observation of deep-sea microorganism samples, and more specifically, to an online purification and sampling and visualized automatic enrichment apparatus and method for deep-sea microorganisms.

### BACKGROUND

The deep sea is considered the last frontier on Earth yet to be fully explored. The extreme conditions of high pressure, low/high temperature, and oligotrophic conditions in the deep-sea environment have given rise to unique deep-sea microorganisms, whose evolutionary branches, biochemical reactions, and metabolic functions remain unsolved mysteries, and possess extremely high research and application value. To investigate the role of deep-sea microorganisms in marine material circulation and the Earth's ecosystem, it is necessary to perform pretreatment, microbial enrichment, and observation on deep-sea environmental samples, thereby identifying their biological community characteristics and physiological metabolic traits. However, due to the low biomass in the deep-sea environment, current technologies for sample treatment and biological enrichment under high-pressure environments are still immature, making rapid enrichment of difficult-to-culture organisms extremely challenging. After deep-sea microorganisms are sampled, it is difficult to purify biological samples under high-pressure environments and monitor biomass changes during the microbial enrichment process, making efficient visualized enrichment of microorganisms very difficult. Therefore, utilizing a temperature- and pressure-retaining apparatus that simulates deep-sea environments for microbial purification and visualized enrichment is an important way to enhance the research level and development efficiency of deep-sea microorganisms.

For purification and sampling of microorganisms under simulated deep-sea conditions, existing patents disclose purification and sampling technologies such as an extracting and semi-quantitative analysis method for biofilms on sediment surfaces and a method for simultaneous purification of nucleic acids and proteins from environmental samples, and these methods mainly involve purification processes such as washing, oscillation, and centrifugation of sediment samples. However, these purification processes are difficult to implement under high-pressure environments. Therefore, it is difficult to separate environmental impurities such as gravels and organic matters from microbial cells in deep-sea environmental samples under high-pressure conditions. There are also limitations such as low activity of the obtained enriched materials, low subsequent biological enrichment efficiency, and low purity of the obtained biological samples.

For enrichment of microorganisms under simulated deep-sea conditions, an existing patent discloses a microbial culture apparatus and system. For the apparatus, the general condition of the internal enriched material is mainly observed through a visualization window and a light-transmitting culture chamber. However, it is difficult to observe the biological abundance of the enriched material in real time under high-pressure environments, and it is still necessary to obtain relative microbial abundance through repeated periodic sampling and gene sequencing analysis. This process involves cumbersome manual operations, low timeliness, and loss of enriched biomass. In addition, an existing patent discloses a high-pressure temperature-controlled online culture and observation sample stage, where an observation window provided on an outlet pipeline of a culture kettle is externally connected to a microscope for real-time observation of microbial morphology. However, samples observed in the pipeline are not diluted, separated, or uniformly leveled, making it difficult to obtain images of interval-separated, flat, and evenly distributed cells for cell counting under the microscope. This technology faces challenges such as excessive cell accumulation in the observed sample, a large number of overlapping cells in the observation field, and no unit grid division in the observation area for cell counting, making it difficult to obtain the actual enriched biomass.

In summary, existing technologies face problems such as difficulty in purifying deep-sea microorganism samples under high-pressure environments, complex manual operations for enrichment and observation, low timeliness of biomass observation, and difficulty in real-time cell counting of enriched materials, which hinder the rapid acquisition of high-purity enriched materials of deep-sea microorganisms.

### SUMMARY

To overcome the shortcomings of the prior art, such as difficulties in purifying deep-sea microorganism samples under high-pressure environments, complex manual operations for enrichment and observation, low timeliness of biomass observation, and difficulty in real-time cell counting of enriched materials, the present invention provides an online purification and sampling and visualized automatic enrichment apparatus and method for deep-sea microorganisms.

Through online sample pretreatment during microorganism sampling, automatic biological concentration and purification, online monitoring of environmental indicators during biological enrichment, online observation of biomass, and automatic enrichment and transfer of high-purity enriched materials, the enrichment rate and acquisition efficiency of difficult-to-culture deep-sea microorganisms are effectively improved, thereby providing a fundamental way for enhancing deep-sea biological research and bioresource development efficiency.

To solve the above technical problems, the technical solutions of the present invention are as follows:
an online purification and sampling apparatus for deep-sea microorganisms is provided, and includes: a sampling and purification unit, a first temperature regulation unit, a first pressure regulation unit, and a first central control unit;
the sampling and purification unit includes: a sample mixing kettle, a sample settling kettle, a sample purification kettle, two sets of pressure-retaining stirrers, a turbidimeter, a total organic carbon analyzer, a purification piston, and a filtration membrane;
the sample mixing kettle is connected to a pipeline of a sample container for acquiring deep-sea microorganism samples from the sample container; a lower part of the sample mixing kettle is connected to an upper pipeline of the sample settling kettle; a lower part of the sample settling kettle is connected to a lower pipeline of the sample purification kettle;
a bottom of the sample settling kettle is of a funnel-shaped structure to achieve sedimentation of impurities in an enriched sample; the two sets of pressure-retaining stirrers are respectively provided at tops of the sample mixing kettle and of the sample purification kettle; the turbidimeter is provided inside the sample settling kettle; the total organic carbon analyzer is connected to the sample purification kettle; the purification piston is provided at a lower part inside the sample purification kettle and is capable of being driven from bottom to top to pressurize concentrated samples; the filtration membrane is provided on an upper surface of the purification piston;
the first temperature regulation unit and the first pressure regulation unit are respectively connected to the first central control unit, the sample mixing kettle, the sample settling kettle, and the sample purification kettle; the first temperature regulation unit and the first pressure regulation unit are respectively controlled by the first central control unit to regulate temperature and pressure inside each of the sample mixing kettle, the sample settling kettle, and the sample purification kettle; and
the first central control unit is further respectively connected to the two sets of pressure-retaining stirrers, the turbidimeter, and the total organic carbon analyzer.

Preferably, the first temperature regulation unit includes: a temperature regulator and three sets of temperature sensors that are each connected to the first central control unit; the three sets of temperature sensors are respectively provided inside the sample mixing kettle, the sample settling kettle, and the sample purification kettle;
the first pressure regulation unit includes: a gas booster pump, three gas injection valves, a purification liquid injection pump, a purification valve, and four sets of pressure sensors; the gas booster pump, the purification liquid injection pump, and four sets of pressure sensors are respectively connected to the first central control unit; and
the gas booster pump is respectively connected to the sample mixing kettle, the sample settling kettle, and the sample purification kettle via the three gas injection valves and the pipeline; the purification liquid injection pump is connected to a bottom of the sample purification kettle via the purification valve and the pipeline; tops of the sample mixing kettle and the sample settling kettle, and the top and bottom of the sample purification kettle are each provided with a set of pressure sensors, wherein the pressure sensor in the bottom of the sample purification kettle is used to monitor a purification progress for immediate stop.

The present invention further provides an online visualized automatic enrichment apparatus for deep-sea microorganisms, and the apparatus includes: an enrichment unit, a visualization monitoring unit, a second temperature regulation unit, a second pressure regulation unit, and a second central control unit;
the enrichment unit includes: an enrichment kettle, a magnetic stirrer, and an environmental parameter sensing system; the magnetic stirrer is provided at a bottom of the enrichment kettle, and a magnetic bar is placed inside the enrichment kettle; the environmental parameter sensing system is respectively connected to the enrichment kettle and the second central control unit for detecting environmental parameters inside the enrichment kettle and transmitting the environmental parameters to the second central control unit;
the visualization monitoring unit includes: a biological observation kettle, a biological abundance measurement system, two sets of pressure-resistant observation windows, a microscopic imaging system, a measurement solenoid valve, and a recovery solenoid valve;
the biological observation kettle is connected to the enrichment kettle via a pipeline; the two sets of pressure-resistant observation windows are respectively provided in a top and bottom of the biological observation kettle and used to observe the morphology of microorganisms inside the biological observation kettle; the biological abundance measurement system is respectively connected to the biological observation kettle and the second central control unit for placing microorganisms to be observed; the microscopic imaging system is provided at the bottom of the biological observation kettle, and connected to the second central control unit and used to observe changes in microbial abundance inside the biological observation kettle through the pressure-resistant observation window at the bottom; the measurement solenoid valve and the recovery solenoid valve are each connected to the biological abundance measurement system and the enrichment kettle via the pipeline respectively, and are respectively connected to the second central control unit; and
the second temperature regulation unit and the second pressure regulation unit are each connected to the second central control unit, the enrichment kettle, and the biological observation kettle respectively; the second temperature regulation unit and the second pressure regulation unit are respectively controlled by the second central control unit to regulate temperature and pressure inside each of the enrichment kettle, and the biological observation kettle.

Preferably, the enrichment unit further includes: an enrichment ball valve, a plunger pump, a continuous liquid injection pump, and a liquid injection valve;
the enrichment ball valve and one end of the plunger pump are respectively connected to the enrichment kettle via the pipeline; the other end of the plunger pump is respectively connected to the measurement solenoid valve and the recovery solenoid valve via the pipeline; the continuous liquid injection pump is sequentially connected to the liquid injection
valve and the enrichment kettle via the pipeline for injecting a microbial culture solution into the enrichment kettle;
the visualization monitoring unit further includes a dilution kettle, a dilution valve, and an illuminator;
one end of the plunger pump is further sequentially connected to the dilution valve and the dilution kettle via the pipeline; a dilution culture solution is accommodated in the dilution kettle; and
the illuminator is provided at the top of the biological observation kettle and connected to the second central control unit for providing illumination to the biological observation kettle from the pressure-resistant observation window in the top.

Preferably, the environmental parameter sensing system includes: a dissolved oxygen detector, a pH detector, and a Raman spectrometer that are respectively used for detecting the dissolved oxygen content, pH value, and changes in chemical substance content inside the enrichment kettle and transmitting the dissolved oxygen content, pH value, and changes in chemical substance content which are detected to the second central control unit.

Preferably, the biological abundance measurement system includes: an observation tank, a cover slip flipper, and a cover slip;
both ends of the observation tank are respectively connected to the measurement solenoid valve and the recovery solenoid valve via the pipeline; the cover slip flipper is connected to the second central control unit for driving the cover slip to flip and attach to the observation tank;
a square groove is provided in the observation tank, and is fully fitted with a preset protruding square on the cover slip; a bidirectional funnel-shaped channel is further provided inside the square groove, and grids for observing and counting cells are engraved in the channel; a plurality of squares of different sizes are provided within the grids for counting cells of different quantities;
the microscopic imaging system includes: a lens switcher and a plurality of microscope lenses of different magnifications; and
the lens switcher is connected to the second central control unit for switching the microscope lenses.

Preferably, the second temperature regulation unit includes: a temperature regulator and two sets of temperature sensors that are each connected to the second central control unit; the two sets of temperature sensors are respectively provided inside the enrichment kettle and the biological observation kettle;
the second pressure regulation unit includes: a gas booster pump, a gas injection valve, a PID control valve, an exhaust valve, and two sets of pressure sensors that are each connected to the second central control unit; and
the gas booster pump is sequentially connected to the gas injection valve and the enrichment kettle via the pipeline; the PID control valve is provided on the enrichment kettle; the exhaust valve is provided on the biological observation kettle; and the two sets of pressure sensors are respectively provided inside the enrichment kettle and the biological observation kettle.

The present invention further provides an online purification and sampling and visualized automatic enrichment apparatus for deep-sea microorganisms, and the apparatus includes a purification and sampling apparatus and an enrichment apparatus sequentially connected via a pipeline, where the purification and sampling apparatus is specifically the online purification and sampling apparatus for deep-sea microorganisms, and the enrichment apparatus is specifically the online visualized automatic enrichment apparatus for deep-sea microorganisms.

The present invention further provides an online purification and sampling and visualized automatic enrichment method for deep-sea microorganisms based on the online purification and sampling and visualized automatic enrichment apparatus for deep-sea microorganisms, and the method includes the following steps:
S1: cleaning and sterilizing a sample mixing kettle, a sample settling kettle, a sample purification kettle, an enrichment kettle, and a biological observation kettle, and respectively injecting a culture solution into the sample mixing kettle, the sample settling kettle, the sample purification kettle, the enrichment kettle, and the biological observation kettle; installing kettle bodies of the sample mixing kettle, the sample settling kettle, the sample purification kettle, the enrichment kettle, and the biological observation kettle and performing gas pressurization on sample mixing kettle, the sample settling kettle, the sample purification kettle, the enrichment kettle, and the biological observation kettle; and installing each unit of the online purification and sampling and visualized automatic enrichment apparatus for deep-sea microorganisms;
S2: acquiring deep-sea microorganism samples from a sample container and transferring the samples to the sample mixing kettle, activating a pressure-retaining stirrer inside the sample mixing kettle, thoroughly mixing the samples after a first preset duration, and obtaining the samples mixed;
S3: transferring the samples mixed to the sample settling kettle, using a turbidimeter to monitor sedimentation and separation of impurities of the samples in real time, and obtaining samples settled when turbidity drops to a first preset threshold;
S4: transferring the samples settled to the sample purification kettle, activating the pressure-retaining stirrer inside the sample purification kettle, driving a purification piston for purification, and using a total organic carbon analyzer to monitor a purification process in real time; when total organic carbon content obtained by the total organic carbon analyzer exceeds a second preset threshold, obtaining purified samples and transferring the purified samples to the enrichment kettle;
S5: activating a magnetic stirrer to initiate enrichment, and using an environmental parameter sensing system to monitor environmental indicators inside the enrichment kettle in real time; when monitored environmental indicators meet preset conditions, obtaining an enriched microbial liquid and completing enrichment;
S6: opening a measurement solenoid valve to transfer the enriched microbial liquid to a biological abundance measurement system inside the biological observation kettle, using a microscopic imaging system for biological abundance observation, and after completing observation, opening a recovery solenoid valve to transfer the enriched microbial liquid observed back to the enrichment kettle for recovery; and
S7: repeating steps S5 and S6, performing multiple rounds of enrichment and observation until observed biological abundance reaches a third preset threshold, and collecting all enriched microbial liquid from the enrichment kettle for further preservation or experimentation.

Preferably, in step S4, when total organic carbon content obtained by the total organic carbon analyzer exceeds a second preset threshold, or when a pressure difference between the pressure sensor at the top and the pressure sensor at the bottom inside the sample purification kettle exceeds a fourth preset threshold, the purified sample is collected.

In the biological abundance measurement system of step S6, a height of the square groove in the observation tank is 0.1 cm, and a grid size is specifically 1 cm×1 cm.

After the microscopic imaging system acquires observation images, the biomass abundance is calculated according to the following formula:

$${N}_{C} = {N}_{F} \times D \times F \times 10000$$

N_{C} represents a quantity of cells per milliliter of sample; N_{F} represents an average quantity of cells in a single grid; D represents a dilution factor of a cell sample; F represents a total quantity of grids of different sizes.

Compared with the prior art, the beneficial effects of the technical solutions of the present invention are as follows:

The present invention provides an online purification and sampling apparatus for deep-sea microorganisms under temperature and pressure retention conditions. Compared with existing high-pressure sampling technologies, the apparatus can, under temperature and pressure retention conditions, achieve an automated pretreatment process for effective separation of biological cells and environmental impurities during microbial sampling under a simulated deep-sea high-pressure environment, as well as acquisition of high-purity biological samples. This solves problems of difficult purification of microbial samples under high-pressure environments and low activity and purity of obtained biological samples during deep-sea microbial sampling, and improves the biological purity and processing efficiency of difficult-to-culture deep-sea microorganisms.

The present invention further provides an online visualized automatic enrichment apparatus for deep-sea microorganisms, and the apparatus addresses the challenges of complex manual operations during biomass observation, low timeliness of observation, and difficulty in real-time cell counting of enriched materials during the enrichment process. In addition, the apparatus enables visualization monitoring of biomass abundance changes after automatic enrichment and purification of microorganisms under deep-sea temperature and pressure retention conditions, thereby improving the acquisition efficiency of high-purity enriched materials of difficult-to-culture deep-sea microorganisms.

In addition, with combination of a purification and sampling technology and an enrichment technology, the present invention provides an online purification and sampling and visualized automatic enrichment apparatus and method for deep-sea microorganisms. Through online sample pretreatment during microorganism sampling, automatic biological concentration and purification, online monitoring of environmental indicators during biological enrichment, online observation of biomass, and automatic enrichment and transfer of high-purity enriched materials, the enrichment rate and acquisition efficiency of difficult-to-culture deep-sea microorganisms are effectively improved, thereby providing a fundamental way for enhancing deep-sea biological research and bioresource development efficiency.

### BRIEF DESCRIPTION OF DRAWINGS

FIG. 1 is an architecture diagram of an online purification and sampling apparatus for deep-sea microorganisms provided in Embodiment 1.

FIG. 2 is an architecture diagram of an online visualized automatic enrichment apparatus for deep-sea microorganisms provided in Embodiment 2.

FIG. 3 is an architecture diagram of an online purification and sampling and visualized automatic enrichment apparatus for deep-sea microorganisms provided in Embodiment 3.

FIG. 4 is a mechanical structure diagram of an online purification and sampling and visualized automatic enrichment apparatus for deep-sea microorganisms provided in Embodiment 4.

FIG. 5 is a detailed structural diagram of a biological observation kettle provided in Embodiment 4.

FIG. 6 is a top view of an internal structure of a biological observation kettle provided in Embodiment 4.

FIG. 7 is a connection diagram of a central control unit provided in Embodiment 4.

FIG. 8 is a flow chart of an online purification and sampling and visualized automatic enrichment method for deep-sea microorganisms provided in Embodiment 5.

FIG. 9 is a detailed flow chart of an online purification and sampling and visualized automatic enrichment method for deep-sea microorganisms provided in Embodiment 5.

### DETAILED DESCRIPTION OF EMBODIMENTS

The drawings are for exemplary illustration only and shall not be construed as limitations to this patent.

To better illustrate this embodiment, certain components in the drawings may be omitted, enlarged, or reduced, and do not represent the dimensions of the actual product.

For those skilled in the art, it is understandable that certain well-known structures and descriptions thereof in the drawings may be omitted.

The technical solutions of the present invention are further illustrated below in conjunction with the drawings and embodiments.

### Embodiment 1

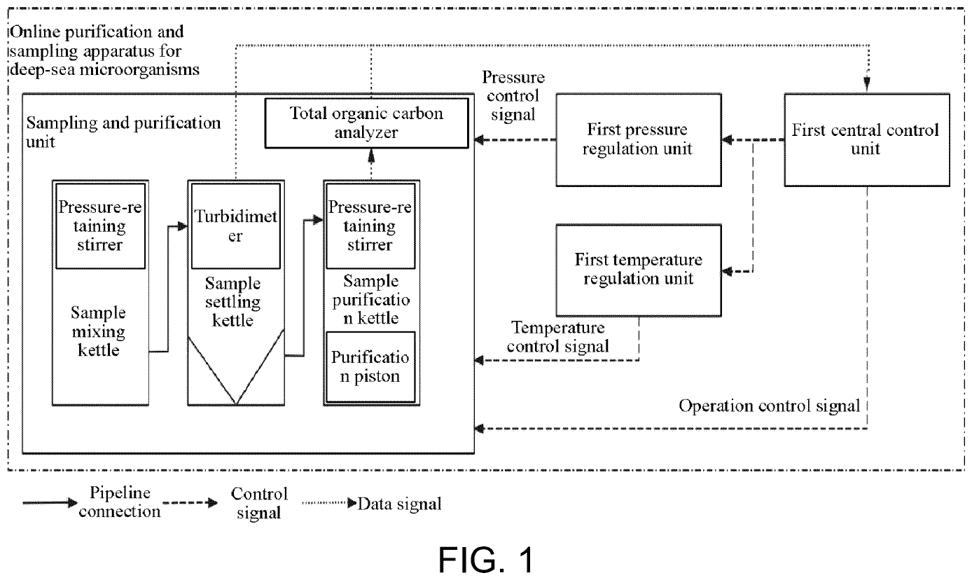

As shown in FIG. 1, this embodiment provides an online purification and sampling apparatus for deep-sea microorganisms, and the apparatus includes: a sampling and purification unit, a first temperature regulation unit, a first pressure regulation unit, and a first central control unit.
the sampling and purification unit includes: a sample mixing kettle, a sample settling kettle, a sample purification kettle, two sets of pressure-retaining stirrers, a turbidimeter, a total organic carbon analyzer, a purification piston, and a filtration membrane;
the sample mixing kettle is connected to a pipeline of a sample container for acquiring deep-sea microorganism samples from the sample container; a lower part of the sample mixing kettle is connected to an upper pipeline of the sample settling kettle; a lower part of the sample settling kettle is connected to a lower pipeline of the sample purification kettle;
a bottom of the sample settling kettle is of a funnel-shaped structure to achieve sedimentation of impurities in an enriched sample; the two sets of pressure-retaining stirrers are respectively provided at tops of the sample mixing kettle and of the sample purification kettle; the turbidimeter is provided inside the sample settling kettle; the total organic carbon analyzer is connected to the sample purification kettle; the purification piston is provided at a lower part inside the sample purification kettle and is capable of being driven from bottom to top to pressurize concentrated samples; the filtration membrane is provided on an upper surface of the purification piston;
the first temperature regulation unit and the first pressure regulation unit are respectively connected to the first central control unit, the sample mixing kettle, the sample settling kettle, and the sample purification kettle; the first temperature regulation unit and the first pressure regulation unit are respectively controlled by the first central control unit to regulate temperature and pressure inside each of the sample mixing kettle, the sample settling kettle, and the sample purification kettle; and
the first central control unit is further respectively connected to the two sets of pressure-retaining stirrers, the turbidimeter, and the total organic carbon analyzer.

In a specific implementation process, a sample mixing kettle, a sample settling kettle, and a sample purification kettle are cleaned and sterilized; a culture solution is respectively injected into the sample mixing kettle, the sample settling kettle, the sample purification kettle, the enrichment kettle, and the biological observation kettle; the kettle bodies of the sample mixing kettle, the sample settling kettle, the sample purification kettle, the enrichment kettle, and the biological observation kettle are installed and gas pressurization is performed on sample mixing kettle, the sample settling kettle, the sample purification kettle, the enrichment kettle, and the biological observation kettle; and each unit of the online purification and sampling apparatus for deep-sea microorganisms is installed.

Deep-sea microorganism samples are acquired from a sample container and are transferred to the sample mixing kettle, a pressure-retaining stirrer inside the sample mixing kettle is activated, the samples are thoroughly mixed after a first preset duration, and the mixed samples are obtained.

The mixed samples are transferred to the sample settling kettle, a turbidimeter is used to monitor sedimentation and separation of impurities in real time, and settled samples are obtained when turbidity drops to a first preset threshold.

The settled samples are transferred to the sample purification kettle, the pressure-retaining stirrer inside the sample purification kettle is activated, a purification piston is driven for purification, and a total organic carbon analyzer is used to monitor a purification process in real time; when total organic carbon content obtained by the total organic carbon analyzer exceeds a second preset threshold, purified samples are obtained and purification and sampling are completed.

The apparatus can, under temperature and pressure retention conditions, achieve an automated pretreatment process for effective separation of biological cells and environmental impurities during microbial sampling under a simulated deep-sea high-pressure environment, as well as acquisition of high-purity biological samples. This solves problems of difficult purification of microbial samples under high-pressure environments and low activity and purity of obtained biological samples during deep-sea microbial sampling, and improves the biological purity and processing efficiency of difficult-to-culture deep-sea microorganisms.

### Embodiment 2

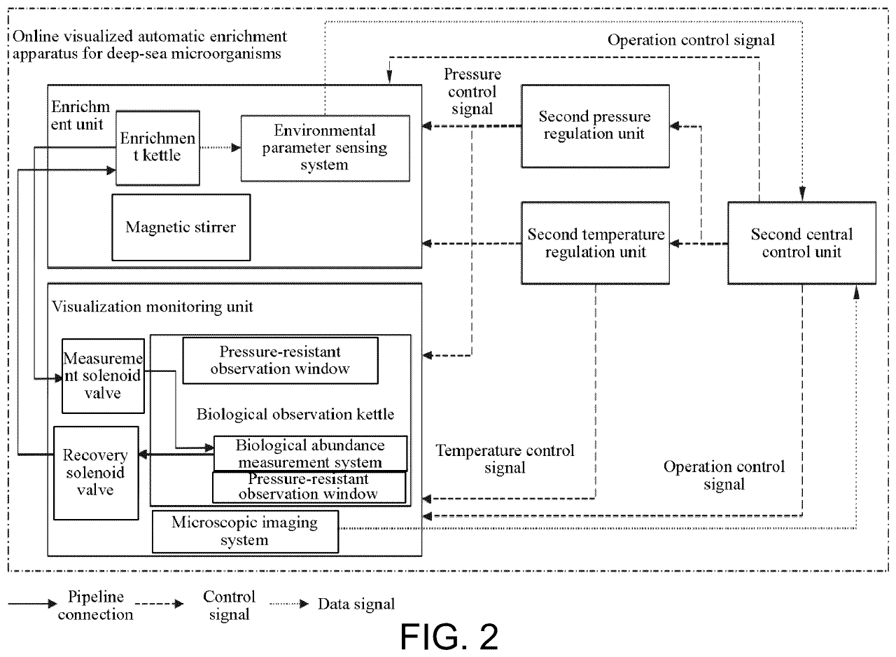

As shown in FIG. 2, this embodiment provides an online visualized automatic enrichment apparatus for deep-sea microorganisms, and the apparatus includes: an enrichment unit, a visualization monitoring unit, a second temperature regulation unit, a second pressure regulation unit, and a second central control unit;
the enrichment unit includes: an enrichment kettle, a magnetic stirrer, and an environmental parameter sensing system; the magnetic stirrer is provided at a bottom of the enrichment kettle, and a magnetic bar is placed inside the enrichment kettle; the environmental parameter sensing system is respectively connected to the enrichment kettle and the second central control unit for detecting environmental parameters inside the enrichment kettle and transmitting the environmental parameters to the second central control unit;
the visualization monitoring unit includes: a biological observation kettle, a biological abundance measurement system, two sets of pressure-resistant observation windows, a microscopic imaging system, a measurement solenoid valve, and a recovery solenoid valve;
the biological observation kettle is connected to the enrichment kettle via a pipeline; the two sets of pressure-resistant observation windows are respectively provided in a top and bottom of the biological observation kettle and used to observe the morphology of microorganisms inside the biological observation kettle; the biological abundance measurement system is respectively connected to the biological observation kettle and the second central control unit for placing microorganisms to be observed; the microscopic imaging system is provided at the bottom of the biological observation kettle, and connected to the second central control unit and used to observe changes in microbial abundance inside the biological observation kettle through the pressure-resistant observation window at the bottom; the measurement solenoid valve and the recovery solenoid valve are each connected to the biological abundance measurement system and the enrichment kettle via the pipeline respectively, and are respectively connected to the second central control unit; and
the second temperature regulation unit and the second pressure regulation unit are each connected to the second central control unit, the enrichment kettle, and the biological observation kettle respectively; the second temperature regulation unit and the second pressure regulation unit are respectively controlled by the second central control unit to regulate temperature and pressure inside each of the enrichment kettle, and the biological observation kettle.

In a specific implementation process, the enrichment kettle and the biological observation kettle are cleaned and sterilized; a culture solution is respectively injected into the sample mixing kettle, the sample settling kettle, the sample purification kettle, the enrichment kettle, and the biological observation kettle; the kettle bodies of the sample mixing kettle, the sample settling kettle, the sample purification kettle, the enrichment kettle, and the biological observation kettle are installed and gas pressurization is performed on sample mixing kettle, the sample settling kettle, the sample purification kettle, the enrichment kettle, and the biological observation kettle; and each unit of the visualized automatic enrichment apparatus for deep-sea microorganisms is installed.

A microbial liquid to be enriched is transferred into the enrichment kettle. A magnetic stirrer is activated to initiate enrichment, and an environmental parameter sensing system is used to monitor environmental indicators inside the enrichment kettle in real time; when monitored environmental indicators meet preset conditions, an enriched microbial liquid is obtained and enrichment is completed.

A measurement solenoid valve is opened to transfer the enriched microbial liquid to a biological abundance measurement system inside the biological observation kettle, a microscopic imaging system is used for biological abundance observation, and after observation, a recovery solenoid valve is opened to transfer an observed enriched microbial liquid back to the enrichment kettle for recovery.

Finally, the above steps are repeated, and multiple rounds of enrichment and observation are performed until observed biomass abundance reaches a third preset threshold. All enriched microbial liquid from the enrichment kettle is collected for further preservation or experimentation.

The apparatus addresses the challenges of complex manual operations during biomass observation, low timeliness of observation, and difficulty in real-time cell counting of enriched materials during the enrichment process. In addition, the apparatus enables visualization monitoring of biomass abundance changes after automatic enrichment and purification of microorganisms under deep-sea temperature and pressure retention conditions, thereby improving the acquisition efficiency of high-purity enriched materials of difficult-to-culture deep-sea microorganisms.

### Embodiment 3

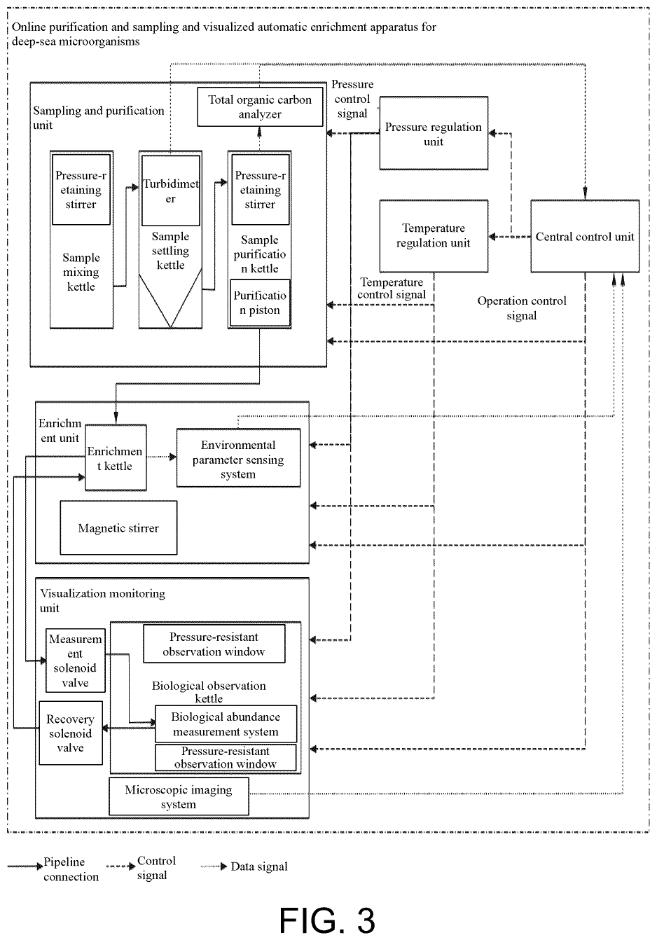

As shown in FIG. 3, this embodiment provides an online purification and sampling and visualized automatic enrichment apparatus for deep-sea microorganisms, and the apparatus includes: a sampling and purification unit, an enrichment unit, a visualization monitoring unit, a temperature regulation unit, a pressure regulation unit, and a central control unit.

In this embodiment, the pressure regulation unit, the temperature regulation unit, and the central control unit are each integrated with the functions of the first pressure regulation unit, the second pressure regulation unit, the first temperature regulation unit, the second temperature regulation unit, the central control unit and the second central control unit, and are respectively used to control the pressure, temperature, and specific operations of the entire apparatus (including the online purification and sampling and enrichment device).

The sampling and purification unit includes: a sample mixing kettle, a sample settling kettle, a sample purification kettle, two sets of pressure-retaining stirrers, a turbidimeter, a total organic carbon analyzer, a purification piston, and a filtration membrane;
the sample mixing kettle is connected to a pipeline of a sample container for acquiring deep-sea microorganism samples from the sample container; a lower part of the sample mixing kettle is connected to an upper pipeline of the sample settling kettle; a lower part of the sample settling kettle is connected to a lower pipeline of the sample purification kettle;
a bottom of the sample settling kettle is of a funnel-shaped structure to achieve sedimentation of impurities in an enriched sample; the two sets of pressure-retaining stirrers are respectively provided at tops of the sample mixing kettle and of the sample purification kettle; the turbidimeter is provided inside the sample settling kettle; the total organic carbon analyzer is connected to the sample purification kettle; the purification piston is provided at a lower part inside the sample purification kettle and is capable of being driven from bottom to top to pressurize concentrated samples; the filtration membrane is provided on an upper surface of the purification piston;
the central control unit is further respectively connected to the two sets of pressure-retaining stirrers, the turbidimeter, and the total organic carbon analyzer;
the enrichment unit includes: an enrichment kettle, a magnetic stirrer, and an environmental parameter sensing system; the magnetic stirrer is provided at a bottom of the enrichment kettle, and a magnetic bar is placed inside the enrichment kettle; the environmental parameter sensing system is respectively connected to the enrichment kettle and the central control unit for detecting environmental parameters inside the enrichment kettle and transmitting the environmental parameters to the central control unit;
the visualization monitoring unit includes: a biological observation kettle, a biological abundance measurement system, two sets of pressure-resistant observation windows, a microscopic imaging system, a measurement solenoid valve, and a recovery solenoid valve;
the biological observation kettle is connected to the enrichment kettle via a pipeline; the two sets of pressure-resistant observation windows are respectively provided in a top and bottom of the biological observation kettle and used to observe the morphology of microorganisms inside the biological observation kettle; the biological abundance measurement system is respectively connected to the biological observation kettle and the central control unit for placing microorganisms to be observed; the microscopic imaging system is provided at the bottom of the biological observation kettle, and connected to the central control unit and used to observe changes in microbial abundance inside the biological observation kettle through the pressure-resistant observation window at the bottom; the measurement solenoid valve and the recovery solenoid valve are each connected to the biological abundance measurement system and the enrichment kettle via the pipeline respectively, and are respectively connected to the central control unit; and
the temperature regulation unit and the pressure regulation unit are respectively connected to the central control unit, the sample mixing kettle, the sample settling kettle, the sample purification kettle, the enrichment kettle, and the biological observation kettle. The temperature regulation unit and the pressure regulation unit are respectively controlled by the central control unit to regulate temperature and pressure inside each of the sample mixing kettle, the sample settling kettle, the sample purification kettle, the enrichment kettle, and the biological observation kettle.

In a specific implementation process, the sample mixing kettle, the sample settling kettle, the sample purification kettle, the enrichment kettle, and the biological observation kettle are cleaned and sterilized; a culture solution is respectively injected into the sample mixing kettle, the sample settling kettle, the sample purification kettle, the enrichment kettle, and the biological observation kettle; the kettle bodies of the sample mixing kettle, the sample settling kettle, the sample purification kettle, the enrichment kettle, and the biological observation kettle are installed and gas pressurization is performed on sample mixing kettle, the sample settling kettle, the sample purification kettle, the enrichment kettle, and the biological observation kettle; and each unit of the apparatus is installed.

Deep-sea microorganism samples are acquired from a sample container and are transferred to the sample mixing kettle, a pressure-retaining stirrer inside the sample mixing kettle is activated, the samples are thoroughly mixed after a first preset duration, and the mixed samples are obtained.

The mixed samples are transferred to the sample settling kettle, a turbidimeter is used to monitor sedimentation and separation of impurities in real time, and settled samples are obtained when turbidity drops to a first preset threshold.

The settled samples are transferred to the sample purification kettle, the pressure-retaining stirrer inside the sample purification kettle is activated, a purification piston is driven for purification, and a total organic carbon analyzer is used to monitor a purification process in real time; when total organic carbon content obtained by the total organic carbon analyzer exceeds a second preset threshold, purified samples are obtained and purification and sampling are completed.

Subsequently, the purified microbial liquid sample is transferred to the enrichment kettle, the magnetic stirrer is activated to initiate enrichment, and an environmental parameter sensing system is used to monitor environmental indicators inside the enrichment kettle in real time. When monitored environmental indicators meet preset conditions, an enriched microbial liquid is obtained, and enrichment is completed.

A measurement solenoid valve is opened to transfer the enriched microbial liquid to a biological abundance measurement system inside the biological observation kettle, a microscopic imaging system is used for biological abundance observation, and after observation, a recovery solenoid valve is opened to transfer an observed enriched microbial liquid back to the enrichment kettle for recovery.

Finally, the above steps are repeated, and multiple rounds of enrichment and observation are performed until observed biomass abundance reaches a third preset threshold. All enriched microbial liquid from the enrichment kettle is collected for further preservation or experimentation.

The apparatus can, under deep-sea temperature and pressure retention conditions, achieve an automated pretreatment process for effective separation of biological cells and environmental impurities during microbial sampling, acquisition of high-purity biological enrichment, real-time detection of biochemical indicators during microbial enrichment, visualization monitoring of biomass, and recycling of the enriched material, thereby effectively improving the sampling purity and enrichment efficiency of deep-sea environmental microorganisms.

### Embodiment 4

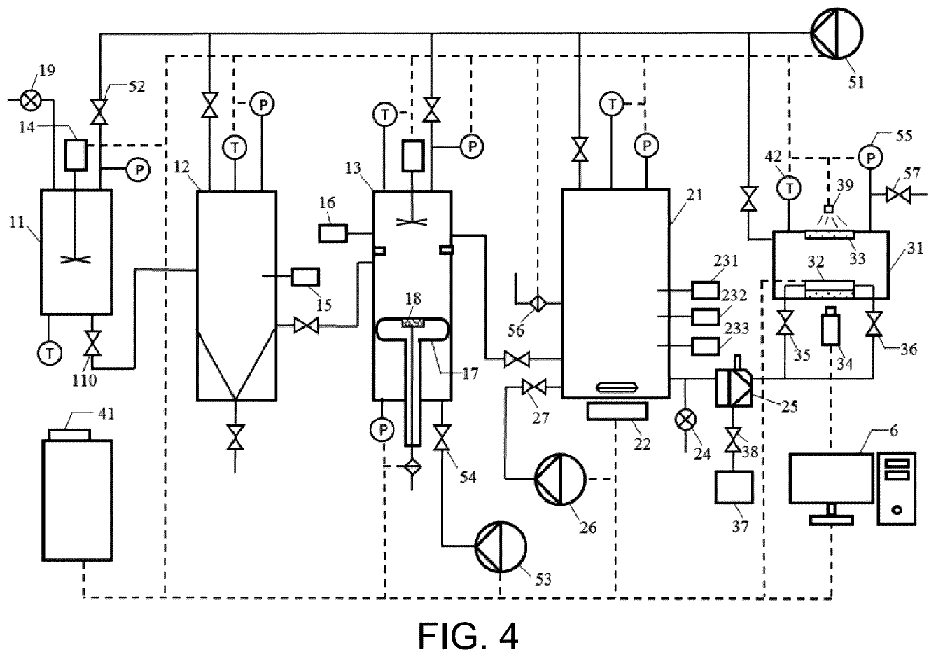

As shown in FIG. 4, this embodiment provides an online purification and sampling and visualized automatic enrichment apparatus for deep-sea microorganisms, and the apparatus includes: a sampling and purification unit 1, an enrichment unit 2, a visualization monitoring unit 3, a temperature regulation unit 4, a pressure regulation unit 5, and a central control unit 6.

The sampling and purification unit 1 includes: a sample mixing kettle 11, a sample settling kettle 12, a sample purification kettle 13, two sets of pressure-retaining stirrers 14, a turbidimeter 15, a total organic carbon analyzer 16, a purification piston 17, and a filtration membrane 18.

The sample mixing kettle 11 is connected to the sample container via the pipeline on which a sampling ball valve 19 is provided, and used to obtain deep-sea microorganism samples from the sampling container; the lower part of the sample mixing kettle 11 is connected to the upper part of the sample settling kettle 12 via the pipeline on which a transfer valve 110 is provided; the lower part of the sample settling kettle 12 is connected to the lower part of the sample purification kettle 13 via the pipeline on which the transfer valve 110 is provided; and the transfer valve 110 is used to facilitate sample transfer during the purification process.

A bottom of the sample settling kettle 12 is of a funnel-shaped structure to achieve sedimentation of impurities in an enriched sample; the two sets of pressure-retaining stirrers 14 are respectively provided at tops of the sample mixing kettle 11 and of the sample purification kettle 13 to achieve sample mixing and mass transfer within the kettle; the turbidimeter 15 is provided inside the sample settling kettle 12 to monitor turbidity changes within the kettle; the total organic carbon analyzer 16 is connected to the sample purification kettle 13 to monitor total organic carbon changes within the kettle; the purification piston 17 is provided below the interior of the sample purification kettle 13 and can be driven from bottom to top to pressurize concentrated samples; and the filtration membrane 18 is provided on an upper surface of the purification piston 17 to filter out the culture solution and increase microbial abundance per unit volume.

Additionally, the upper half part of the sample purification kettle 13 in this embodiment is provided with a protruding circular ring 111 with a smaller inner diameter, and the protruding circular ring 111 is integrated with the sample purification kettle 13 to prevent the purification piston 17 from being driven by increased pressure at the bottom thereof and damaging the pressure-retaining stirrer 14 at the top and the interface of the total organic carbon analyzer 17.

The central control unit 6 is further respectively connected to the two sets of pressure-retaining stirrers 14, the turbidimeter 15, and the total organic carbon analyzer 16.

The enrichment unit 2 includes: an enrichment kettle 21, a magnetic stirrer 22, and an environmental parameter sensing system 23; the magnetic stirrer 22 is provided at a bottom of the enrichment kettle 21, and a magnetic bar is placed inside the enrichment kettle to achieve stirring of the enriched materials and enhance mass transfer; the environmental parameter sensing system 23 is respectively connected to the enrichment kettle 21 and the central control unit 6 for detecting environmental parameters inside the enrichment kettle 21 and transmitting the environmental parameters to the central control unit 6, so as to periodically measure biochemical processes within the kettle and assess enrichment effectiveness.

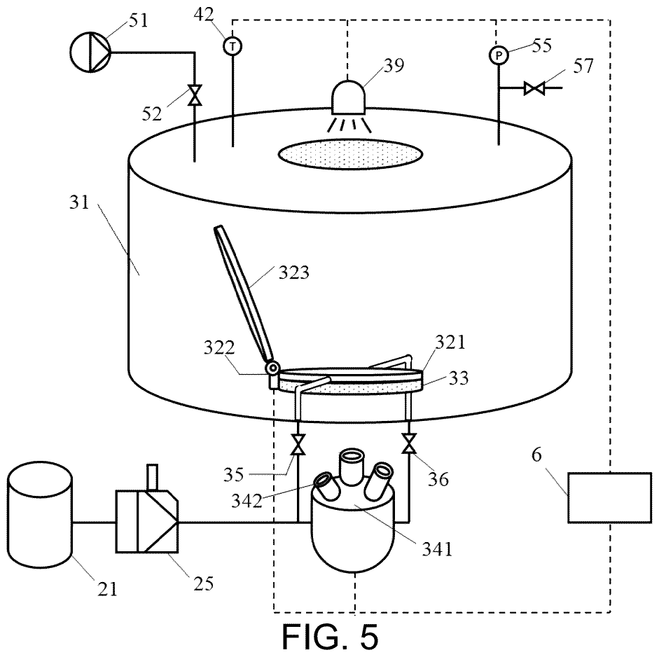

As shown in FIG. 5, the visualization monitoring unit 3 includes: a biological observation kettle 31, a biological abundance measurement system 32, two sets of pressure-resistant observation windows 33, a microscopic imaging system 34, a measurement solenoid valve 35, and a recovery solenoid valve 36.

The biological observation kettle 31 is connected to the enrichment kettle 21 via a pipeline; the two sets of pressure-resistant observation windows 33 are respectively provided on top and bottom surfaces of the biological observation kettle 31 in the biological observation kettle 31 and made of pressure-resistant, high-transparency material, and used to observe the morphology of microorganisms inside the biological observation kettle 31; the biological abundance measurement system 32 is respectively connected to the biological observation kettle 31 and the central control unit 6 for placing microorganisms to be observed; the microscopic imaging system 34 is provided at the bottom of the biological observation kettle 31, and connected to the central control unit 6 and used to observe changes in microbial abundance inside the biological observation kettle 31 through the pressure-resistant observation window 33 at the bottom; and the measurement solenoid valve 35 and the recovery solenoid valve 36 are each connected to the biological abundance measurement system 32 and the enrichment kettle 21 via the pipeline respectively, and are respectively connected to the central control unit 6. The measurement solenoid valve 35 and the recovery solenoid valve 36 are used for sample injection, dilution, and recovery during observation.

The temperature regulation unit 4 and the pressure regulation unit 5 are respectively connected to the central control unit 6, the sample mixing kettle 11, the sample settling kettle 12, the sample purification kettle 13, the enrichment kettle 21, and the biological observation kettle 31. The temperature regulation unit 4 and the pressure regulation unit 5 are respectively controlled by the central control unit 6 to regulate temperature and pressure inside each of the sample mixing kettle 11, the sample settling kettle 12, the sample purification kettle 13, the enrichment kettle 21, and the biological observation kettle 31.

In this embodiment, the enrichment unit 2 further includes: an enrichment ball valve 24, a plunger pump 25, a continuous liquid injection pump 26, and a liquid injection valve 27.

The enrichment ball valve 24 and one end of the plunger pump 25 are respectively connected to the enrichment kettle 21 via the pipeline for collecting the enriched material after culture; the other end of the plunger pump 25 is respectively connected to the measurement solenoid valve 35 and the recovery solenoid valve 36 via the pipeline;
the continuous liquid injection pump 26 is sequentially connected to the liquid injection valve 27 and the enrichment kettle 21 via the pipeline for injecting a microbial culture solution into the enrichment kettle 21;
the visualization monitoring unit 3 further includes a dilution kettle 37, a dilution valve 38, and an illuminator 39; and
one end of the plunger pump 25 is further sequentially connected to the dilution valve 38 and the dilution kettle 37 via the pipeline; a dilution culture solution is accommodated in the dilution kettle 37 and injected into the observation tank 321 and used to dilute the enriched material during biological observation, thereby facilitating better observation and counting of biological cells.

The illuminator 39 is provided at the top of the biological observation kettle 31 and connected to the central control unit 6 for providing illumination to the biological observation kettle 31 from the pressure-resistant observation window 33 in the top.

The environmental parameter sensing system 23 includes: a dissolved oxygen detector 231, a pH detector 232, and a Raman spectrometer 233 that are respectively used for detecting the dissolved oxygen content, pH value, and changes in chemical substance content inside the enrichment kettle 21 and transmitting the dissolved oxygen content, pH value, and changes in chemical substance content which are detected to the central control unit 6.

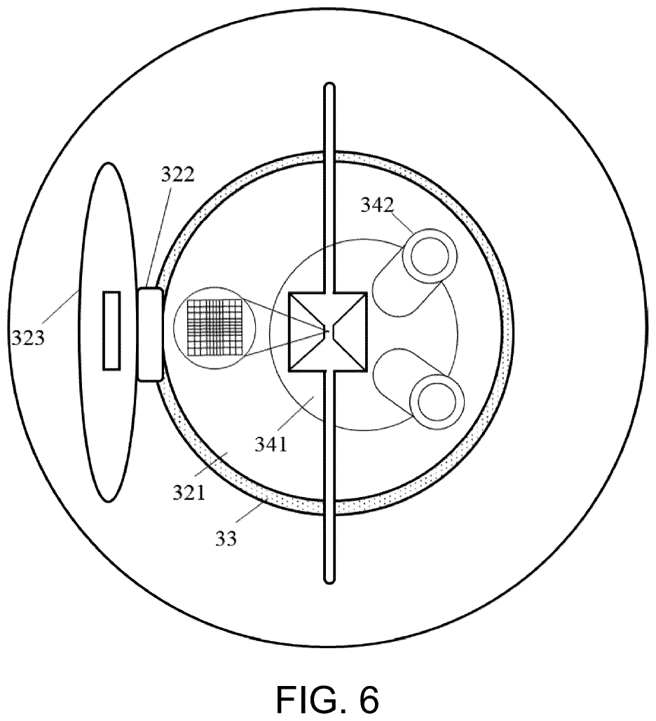

As shown in FIG. 6, the biological abundance measurement system 32 includes: an observation tank 321, a cover slip flipper 322, and a cover slip 323; the observation tank 321 and the cover slip 323 are both made of high-transparency material, and when the observation tank and the cover slip are fitted together, the enriched microbial liquid sample is allowed to remain between them for microscopic observation.

Both ends of the observation tank 321 are respectively connected to the measurement solenoid valve 35 and the recovery solenoid valve 36 via the pipeline for sample injection and recovery; and the cover slip flipper 322 is connected to the central control unit 6 for driving the cover slip 323 to flip and attach to the observation tank 321.

A square groove is provided in the observation tank 321, and is fully fitted with a preset protruding square on the cover slip 323; a bidirectional funnel-shaped channel is further provided inside the square groove, and grids for observing and counting cells are engraved in the channel; in this embodiment, the actual volume of the microbial liquid accommodated in a grid area is approximately 10 μL, that is, a dimension is 1×1×0.01 cm³. The grid contains four large squares around the periphery and one small square in the center, which are used to count cells of different quantities. When a quantity of cells is small, the large squares are used for counting; and when the quantity of cells is large, the small squares are used for counting.

The microscopic imaging system 34 includes: a lens switcher 341 and three microscope lenses 342 with different magnifications. In this embodiment, the magnifications of the microscope lenses include 50×, 100×, and 200×, so as to enable multiple field-of-view switching during biomass observation.

The lens switcher 341 is connected to the central control unit 6 for switching the microscope lenses 342.

In addition, in this embodiment, the microscopic imaging system 34 is further equipped with image analysis software connected to the central control unit 6, which can automatically identify and count cells. The counting rule is represented by the following formula:

$${N}_{C} = {N}_{F} \times D \times F \times 10000$$

NC represents a quantity of cells per milliliter of sample; NF represents an average quantity of cells in a grid; D represents a dilution factor of a cell sample; F represents a quantity of grids, including quantities of two types of grids, with 16 large grids and 25 small grids.

The temperature regulation unit 4 includes: a temperature regulator 41 and five sets of temperature sensors 42 that are each connected to the central control unit 6; the five sets of temperature sensors 42 are respectively provided inside the sample mixing kettle 11, the sample settling kettle 12, the sample purification kettle 13, the enrichment kettle 21, and the biological observation kettle 31.

The pressure regulation unit 5 includes: a gas booster pump 51, four gas injection valves 52, a purification liquid injection pump 53, a purification valve 54, six sets of pressure sensors 55, a PID control valve 56, and an exhaust valve 57; and the gas booster pump 51, the purification liquid injection pump 53, and all pressure sensors 55 are each connected to the central control unit 6.

The gas booster pump 51 is respectively connected to the sample mixing kettle 11, the sample settling kettle 12, the sample purification kettle 13, and the enrichment kettle 21 via four gas injection valves 52 and the pipeline, so as to provide gas pressure; the purification liquid injection pump 53 is connected to the bottom of the sample purification kettle 13 via the purification valve 54 and the pipeline, so as to provide liquid pressure during purification; tops of the sample mixing kettle 11 and the sample settling kettle 12, and the top and bottom of the sample purification kettle 13 are each provided with a set of pressure sensors 55, where the pressure sensor 55 in the bottom of the sample purification kettle 13 is used to monitor a purification progress for immediate stop; the pressure sensors 55 are also provided inside the enrichment kettle 21 and the biological observation kettle 31; the PID control valve 56 is provided on the enrichment kettle 21 to maintain continuous environments and pressure inside the kettle; and the exhaust valve 57 is provided on the biological observation kettle 31 to control the discharge of high-pressure gas.

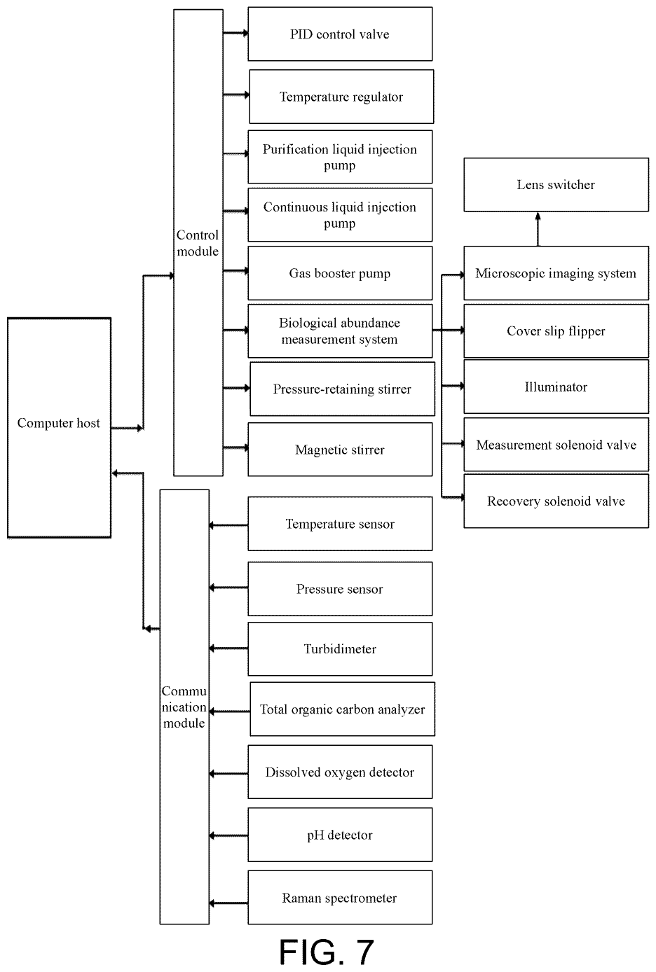

As shown in FIG. 7, in this embodiment, the central control unit 6 includes a communication module, a control module, and a computer host. The communication module is used to connect various sensors and acquire changes in environmental parameters; the communication module is connected and in communication with the computer host via a circuit; the control module is connected to each operating device, and the control module is connected to the computer host via the circuit. The computer host sends electrical signals to control the functions of devices in the control module, including pressure regulation, temperature regulation, filtration and purification, continuous regulation, mixing and mass transfer, and abundance observation; and the computer host monitors and controls all electronic instruments in the device via the communication module and the control module.

In a specific implementation process, the sample mixing kettle 11, the sample settling kettle 12, the sample purification kettle 13, the enrichment kettle 21, and the biological observation kettle 31 are cleaned and sterilized; a culture solution is respectively injected into the sample mixing kettle, the sample settling kettle, the sample purification kettle, the enrichment kettle, and the biological observation kettle; the kettle bodies of the sample mixing kettle, the sample settling kettle, the sample purification kettle, the enrichment kettle, and the biological observation kettle are installed and gas pressurization is performed on sample mixing kettle, the sample settling kettle, the sample purification kettle, the enrichment kettle, and the biological observation kettle; and each unit of the apparatus is installed.

Deep-sea microorganism samples are acquired from a sample container and are transferred to the sample mixing kettle 11, a pressure-retaining stirrer 14 inside the sample mixing kettle 11 is activated, the samples are thoroughly mixed after a first preset duration, and the mixed samples are obtained.

The mixed samples are transferred to the sample settling kettle 12, a turbidimeter 15 is used to monitor sedimentation and separation of impurities in real time, and settled samples are obtained when turbidity drops to a first preset threshold.

The settled samples are transferred to the sample purification kettle 13, the pressure-retaining stirrer 14 inside the sample purification kettle 13 is activated, a purification piston 17 is driven for purification, and a total organic carbon analyzer 16 is used to monitor a purification process in real time; when total organic carbon content obtained by the total organic carbon analyzer 16 exceeds a second preset threshold, or when a pressure difference between the pressure sensor 55 at the top and the pressure sensor 55 at the bottom inside the sample purification kettle 13 exceeds a fourth preset threshold, purified samples are obtained and purification and sampling are completed.

Subsequently, the purified microbial liquid sample is transferred to the enrichment kettle 21, the magnetic stirrer 22 is activated to initiate enrichment, and an environmental parameter sensing system 23 is used to monitor environmental indicators inside the enrichment kettle 21 in real time. When monitored environmental indicators meet preset conditions, an enriched microbial liquid is obtained, and enrichment is completed.

When the enrichment environment meets requirements, the cover slip flipper 322 is activated to flip open the cover slip 323, the measurement solenoid valve 35 is opened, and the plunger pump 25 is connected to the enrichment kettle 21 to inject an appropriate amount of enriched microbial liquid into the biological observation kettle 31.

The dilution valve 38 is opened, the plunger pump 25 is connected to the dilution kettle 37, and a dilution culture solution is injected into the biological observation kettle 31.

The cover slip flipper 322 is controlled to close the cover slip 323, and the measurement solenoid valve 35 is closed; the microscopic imaging system 34 automatically identifies and calculates a quantity of cells per unit area in the grid during image acquisition.

After each observation is completed, the recovery solenoid valve 36 is opened, the plunger pump 25 is connected to the biological observation kettle 31 to inject the enriched microbial liquid into the enrichment kettle 21 at a specified flow rate, and the observed microbial liquid is recovered to the enrichment kettle 21. The recovery solenoid valve 36 is then closed, and the total biomass is calculated using the image recognition software of the microscopic imaging system 34.

The above steps are repeated, and multiple rounds of observations are performed until a specified biological abundance is reached. The enrichment ball valve 24 is then opened to collect all enriched materials in the enrichment kettle 21 for further preservation and experimentation.

The apparatus can, under deep-sea temperature and pressure retention conditions, achieve an automated pretreatment process for effective separation of biological cells and environmental impurities during microbial sampling, acquisition of high-purity biological enrichment, real-time detection of biochemical indicators during microbial enrichment, visualization monitoring of biomass, and recycling of the enriched material, thereby effectively improving the sampling purity and enrichment efficiency of deep-sea environmental microorganisms.

### Embodiment 5

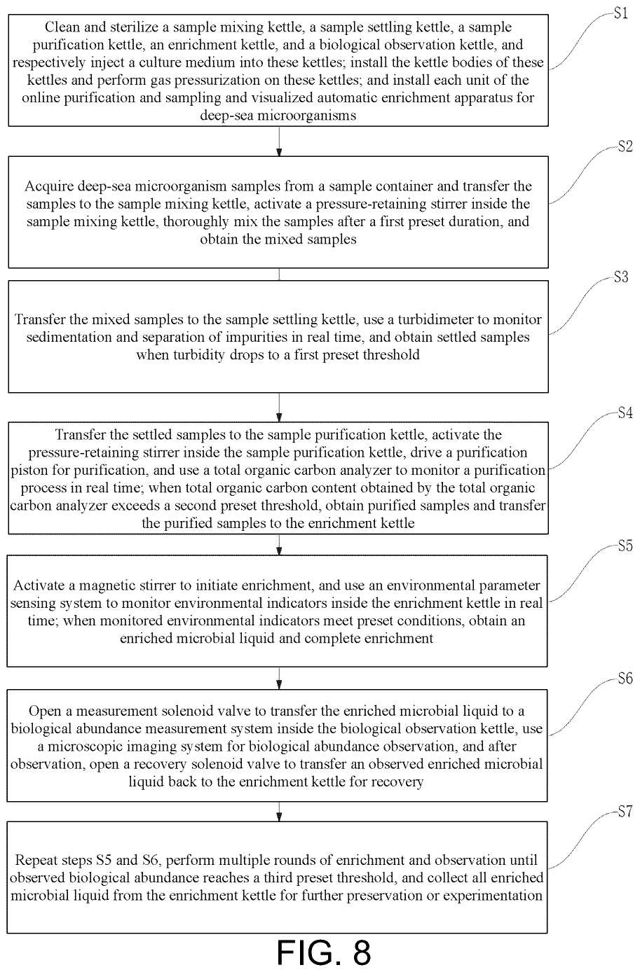

As shown in FIG. 8, this embodiment provides an online purification and sampling and visualized automatic enrichment method for deep-sea microorganisms based on the online purification and sampling and visualized automatic enrichment apparatus for deep-sea microorganisms described in Embodiment 4, and the method includes the following steps:
S1: clean and sterilize a sample mixing kettle, a sample settling kettle, a sample purification kettle, an enrichment kettle, and a biological observation kettle, and respectively inject a culture solution into the sample mixing kettle, the sample settling kettle, the sample purification kettle, the enrichment kettle, and the biological observation kettle; install the kettle bodies of the sample mixing kettle, the sample settling kettle, the sample purification kettle, the enrichment kettle, and the biological observation kettle and perform gas pressurization on sample mixing kettle, the sample settling kettle, the sample purification kettle, the enrichment kettle, and the biological observation kettle; and install each unit of the online purification and sampling and visualized automatic enrichment apparatus for deep-sea microorganisms;
S2: acquire deep-sea microorganism samples from a sample container and transfer the samples to the sample mixing kettle, activate a pressure-retaining stirrer inside the sample mixing kettle, thoroughly mix the samples after a first preset duration, and obtain the samples mixed;
S3: transfer the samples mixed to the sample settling kettle, use a turbidimeter to monitor sedimentation and separation of impurities of the samples in real time, and obtain samples settled when turbidity drops to a first preset threshold;
S4: transfer the samples settled to the sample purification kettle, activate the pressure-retaining stirrer inside the sample purification kettle, drive a purification piston for purification, and use a total organic carbon analyzer to monitor a purification process of the samples in real time; when total organic carbon content obtained by the total organic carbon analyzer exceeds a second preset threshold, or when a pressure difference between the pressure sensor at the top and the pressure sensor at the bottom inside the sample purification kettle exceeds a fourth preset threshold, obtain the samples purified and transfer the samples purified to the enrichment kettle;
S5: activate a magnetic stirrer to initiate enrichment, and use an environmental parameter sensing system to monitor environmental indicators inside the enrichment kettle in real time; when monitored environmental indicators meet preset conditions, obtain an enriched microbial liquid and complete enrichment;
S6: open a measurement solenoid valve to transfer the enriched microbial liquid to a biological abundance measurement system inside the biological observation kettle, use a microscopic imaging system for biological abundance observation, and after observation, open a recovery solenoid valve to transfer an observed enriched microbial liquid back to the enrichment kettle for recovery; and
S7: repeat steps S5 and S6, perform multiple rounds of enrichment and observation until observed biological abundance reaches a third preset threshold, and collect all enriched microbial liquid from the enrichment kettle for further preservation or experimentation.

In the biological abundance measurement system of step S6, a height of the square groove in the observation tank is 0.1 cm, and a grid size is specifically 1 cm×1 cm.

After the microscopic imaging system acquires observation images, the biomass abundance is calculated according to the following formula:

$${N}_{C} = {N}_{F} \times D \times F \times 10000$$

N_{C} represents a quantity of cells per milliliter of sample; N_{F} represents an average quantity of cells in a single grid; D represents a dilution factor of a cell sample; F represents a total quantity of grids of different sizes.

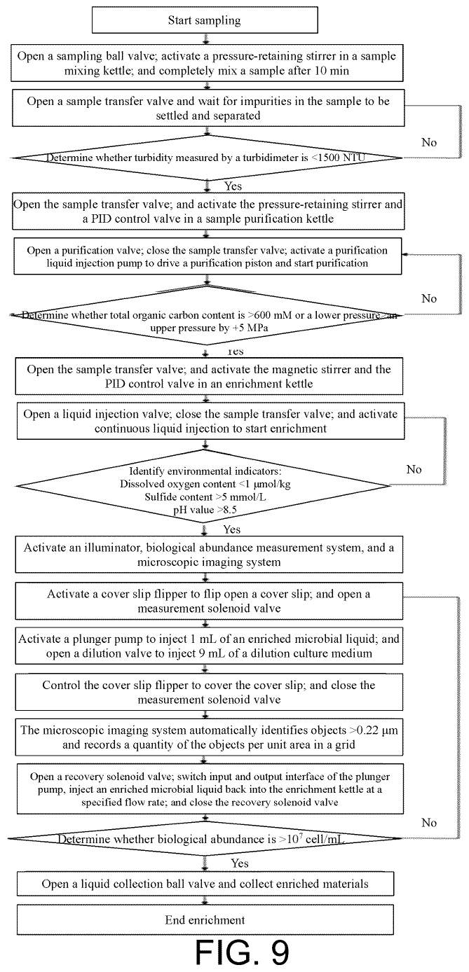

In a specific implementation process, as shown in FIG. 9, the control method includes the following steps:

After the sample mixing kettle, the sample settling kettle, the sample purification kettle, the enrichment kettle, and the biological observation kettle are filled with a culture solution and high-pressure gas, connect the sample mixing kettle 11 to a front-end simulation apparatus, start sampling, open the sampling ball valve 19, activate the pressure-retaining stirrer 14 of the sample mixing kettle 11, and thoroughly mix the sample and the culture solution after 10 min.

When the sample is thoroughly mixed, open the sample transfer valve 110 behind the sample mixing kettle 11, transfer the sample to the sample settling kettle 12, and wait for sedimentation and separation of impurities and the enriched material, identify the separation status by the turbidimeter 15. If the turbidity measured by the turbidimeter 15 is <1500 NTU, open the sample transfer valve 110 behind the sample settling kettle 12, and activate the pressure-retaining stirrer 14 and the PID control valve 56 in the sample purification kettle 13; otherwise, continue waiting for sedimentation and separation.

After the settled sample is transferred, open the purification valve 54, close the sample transfer valve 110, activate the purification liquid injection pump 53, drive the purification piston 17, start purification, and identify the purification status by the total organic carbon analyzer 16. If the total organic carbon content measured by the total organic carbon analyzer 16 is >600 mM or the lower pressure indicated by the two pressure sensors 55 in the sample purification kettle 13 is >the upper pressure +5 MPa, open the sample transfer valve 110 behind the sample purification kettle 13, and activate the magnetic stirrer 22 and PID control valve 56 in the enrichment kettle 21; otherwise, continue the above purification operation.

After sample purification is completed, open the liquid injection valve 27, close the sample transfer valve 110 behind the sample purification kettle 13, activate the continuous liquid injection pump 26, start enrichment, and identify environmental indicators inside the enrichment kettle 21 by the dissolved oxygen detector 231, the pH detector 232, and the Raman spectrometer 233. If the dissolved oxygen content is <1 μmol/kg, sulfide content is >5 mmol/L, and pH value is >8.5, determine that biological observation can start. Activate the illuminator 39 of the biological observation kettle 31, the biological abundance measurement system 32, and the microscopic imaging system 34; otherwise, continue waiting for the enrichment environment to meet the requirements.

When the enrichment environment meets the requirements, start biological observation. Activate the cover slip flipper 322 to flip open the cover slip 323, and open the measurement solenoid valve 35; activate the plunger pump 25, connect the plunger pump to the enrichment kettle 21, and inject 0.1 mL of enriched microbial liquid into the biological observation kettle 31; open the dilution valve 38, connect the plunger pump 25 to the dilution kettle 37, and inject 0.9 mL of dilution culture solution into the biological observation kettle 31; control the cover slip flipper 322 to close the cover slip 323, and close the measurement solenoid valve 35; automatically identify objects <0.22 μm in the acquired image by the microscopic imaging system 34 and record a quality of cells per unit area in the grid.

After each observation is completed, open the recovery solenoid valve 36, connect the plunger pump 25 to the biological observation kettle 31, inject the observed enriched microbial liquid into the enrichment kettle 21 at a specified flow rate for recovery; then close the recovery solenoid valve 36; and calculate the total biomass using the image recognition software of the microscopic imaging system 34. If the biological abundance is >107 cell/mL, open the enrichment ball valve 24 to collect the enriched material and end enrichment; otherwise, continue the above enrichment process until the enriched material with high biological abundance biomass is obtained.

Identical or similar reference numerals correspond to identical or similar components.

Terms describing positional relationships in the drawings are for exemplary illustration only and shall not be construed as limitations to this patent.

Obviously, the above embodiments of the present invention are merely examples to clearly illustrate the present invention and are not intended to limit the implementations of the present invention. For those of ordinary skill in the art, other variations or modifications in different forms can be made based on the above description. It is neither necessary nor possible to exhaustively list all implementations herein. Any modifications, equivalent substitutions, improvements, and the like made within the spirit and principles of the present invention shall be included within the scope of protection of the claims of the present invention.

1. An online purification and sampling apparatus for deep-sea microorganisms, comprising: a sampling and purification unit, a first temperature regulation unit, a first pressure regulation unit, and a first central control unit;
wherein the sampling and purification unit comprises: a sample mixing kettle, a sample settling kettle, a sample purification kettle, two sets of pressure-retaining stirrers, a turbidimeter, a total organic carbon analyzer, a purification piston, and a filtration membrane;
wherein the sample mixing kettle is connected to a pipeline of a sample container for acquiring deep-sea microorganism samples from the sample container; a lower part of the sample mixing kettle is connected to an upper pipeline of the sample settling kettle; a lower part of the sample settling kettle is connected to a lower pipeline of the sample purification kettle;
wherein a bottom of the sample settling kettle is of a funnel-shaped structure to achieve sedimentation of impurities in an enriched sample; the two sets of pressure-retaining stirrers are respectively provided at tops of the sample mixing kettle and of the sample purification kettle; the turbidimeter is provided inside the sample settling kettle; the total organic carbon analyzer is connected to the sample purification kettle; the purification piston is provided at a lower part inside the sample purification kettle and is capable of being driven from bottom to top to pressurize concentrated samples; the filtration membrane is provided on an upper surface of the purification piston;
wherein the first temperature regulation unit and the first pressure regulation unit are each connected to the first central control unit, the sample mixing kettle, the sample settling kettle, and the sample purification kettle respectively; the first temperature regulation unit and the first pressure regulation unit are respectively controlled by the first central control unit to regulate temperature and pressure inside each of the sample mixing kettle, the sample settling kettle, and the sample purification kettle; and
wherein the first central control unit is further respectively connected to the two sets of pressure-retaining stirrers, the turbidimeter, and the total organic carbon analyzer.

2. The online purification and sampling apparatus for deep-sea microorganisms according to claim 1, wherein the first temperature regulation unit comprises: a temperature regulator and three sets of temperature sensors that are each connected to the first central control unit; the three sets of temperature sensors are respectively provided inside the sample mixing kettle, the sample settling kettle, and the sample purification kettle;
the first pressure regulation unit comprises: a gas booster pump, three gas injection valves, a purification liquid injection pump, a purification valve, and four sets of pressure sensors; the gas booster pump, the purification liquid injection pump, and the four sets of pressure sensors are respectively connected to the first central control unit; and
the gas booster pump is respectively connected to the sample mixing kettle, the sample settling kettle, and the sample purification kettle via the three gas injection valves and the pipeline; the purification liquid injection pump is connected to a bottom of the sample purification kettle via the purification valve and the pipeline; tops of the sample mixing kettle and the sample settling kettle, and the top and bottom of the sample purification kettle are each provided with a set of pressure sensors, wherein the pressure sensor in the bottom of the sample purification kettle is used to monitor a purification progress for immediate stop.

3. An online visualized automatic enrichment apparatus for deep-sea microorganisms, comprising: an enrichment unit, a visualization monitoring unit, a second temperature regulation unit, a second pressure regulation unit, and a second central control unit;
the enrichment unit comprises: an enrichment kettle, a magnetic stirrer, and an environmental parameter sensing system; the magnetic stirrer is provided at a bottom of the enrichment kettle, and a magnetic bar is placed inside the enrichment kettle; the environmental parameter sensing system is respectively connected to the enrichment kettle and the second central control unit for detecting environmental parameters inside the enrichment kettle and transmitting the environmental parameters to the second central control unit;
the visualization monitoring unit comprises: a biological observation kettle, a biological abundance measurement system, two sets of pressure-resistant observation windows, a microscopic imaging system, a measurement solenoid valve, and a recovery solenoid valve;
the biological observation kettle is connected to the enrichment kettle via a pipeline; the two sets of pressure-resistant observation windows are respectively provided in a top and a bottom of the biological observation kettle, and used to observe morphology of microorganisms inside the biological observation kettle; the biological abundance measurement system is respectively connected to the biological observation kettle and the second central control unit for placing microorganisms to be observed; the microscopic imaging system is provided at the bottom of the biological observation kettle and connected to the second central control unit for observing changes in microbial abundance inside the biological observation kettle through the pressure-resistant observation window in the bottom; the measurement solenoid valve and the recovery solenoid valve are each connected to the biological abundance measurement system and the enrichment kettle via the pipeline respectively, and are respectively connected to the second central control unit; and
the second temperature regulation unit and the second pressure regulation unit are each connected to the second central control unit, the enrichment kettle, and the biological observation kettle respectively; the second temperature regulation unit and the second pressure regulation unit are respectively controlled by the second central control unit to regulate temperature and pressure inside each of the enrichment kettle, and the biological observation kettle.

4. The online visualized automatic enrichment apparatus for deep-sea microorganisms according to claim 3, wherein the enrichment unit further comprises: an enrichment ball valve, a plunger pump, a continuous liquid injection pump, and a liquid injection valve;
the enrichment ball valve and one end of the plunger pump are respectively connected to the enrichment kettle via the pipeline; an other end of the plunger pump is respectively connected to the measurement solenoid valve and the recovery solenoid valve via the pipeline;
the continuous liquid injection pump is sequentially connected to the liquid injection valve and the enrichment kettle via the pipeline for injecting a microbial culture solution into the enrichment kettle;
the visualization monitoring unit further comprises a dilution kettle, a dilution valve, and an illuminator;
one end of the plunger pump is further sequentially connected to the dilution valve and the dilution kettle via the pipeline; a dilution culture solution is accommodated in the dilution kettle; and
the illuminator is provided at the top of the biological observation kettle and connected to the second central control unit for providing illumination to the biological observation kettle from the pressure-resistant observation window in a top.

5. The online visualized automatic enrichment apparatus for deep-sea microorganisms according to claim 3, wherein the environmental parameter sensing system comprises: a dissolved oxygen detector, a pH detector, and a Raman spectrometer that are respectively used for detecting the dissolved oxygen content, pH value, and changes in chemical substance content inside the enrichment kettle and transmitting the dissolved oxygen content, pH value, and changes in chemical substance content which are detected to the second central control unit.

6. The online visualized automatic enrichment apparatus for deep-sea microorganisms according to claim 3, wherein the biological abundance measurement system comprises: an observation tank, a cover slip flipper, and a cover slip;
both ends of the observation tank are respectively connected to the measurement solenoid valve and the recovery solenoid valve via the pipeline; the cover slip flipper is connected to the second central control unit for driving the cover slip to flip and attach to the observation tank;
a square groove is provided in the observation tank, and is fully fitted with a preset protruding square on the cover slip; a bidirectional funnel-shaped channel is further provided inside the square groove, and grids for observing and counting cells are engraved in the channel; a plurality of squares of different sizes are provided within the grids for counting cells of different quantities;
the microscopic imaging system comprises: a lens switcher and a plurality of microscope lenses of different magnifications; and
the lens switcher is connected to the second central control unit for switching the microscope lenses.

7. The online visualized automatic enrichment apparatus for deep-sea microorganisms according to claim 3, wherein the second temperature regulation unit comprises: a temperature regulator and two sets of temperature sensors that are each connected to the second central control unit; the two sets of temperature sensors are respectively provided inside the enrichment kettle and the biological observation kettle;
the second pressure regulation unit comprises: a gas booster pump, a gas injection valve, a PID control valve, an exhaust valve, and two sets of pressure sensors that are each connected to the second central control unit; and
the gas booster pump is sequentially connected to the gas injection valve and the enrichment kettle via the pipeline; the PID control valve is provided on the enrichment kettle; the exhaust valve is provided on the biological observation kettle; and the two sets of pressure sensors are respectively provided inside the enrichment kettle and the biological observation kettle.

8. An online purification and sampling and visualized automatic enrichment apparatus for deep-sea microorganisms, comprising a purification and sampling apparatus and an enrichment apparatus sequentially connected via a pipeline, wherein the purification and sampling apparatus is the online purification and sampling apparatus for deep-sea microorganisms according to claim 1, and the enrichment apparatus is the online visualized automatic enrichment apparatus for deep-sea microorganisms according to claim 3.

9. An online purification and sampling and visualized automatic enrichment method for deep-sea microorganisms based on the online purification and sampling and visualized automatic enrichment apparatus for deep-sea microorganisms according to claim 8, comprising the following steps:
S1: cleaning and sterilizing the sample mixing kettle, the sample settling kettle, the sample purification kettle, the enrichment kettle, and the biological observation kettle, and respectively injecting a culture solution into the sample mixing kettle, the sample settling kettle, the sample purification kettle, the enrichment kettle, and the biological observation kettle; installing kettle bodies of the sample mixing kettle, the sample settling kettle, the sample purification kettle, the enrichment kettle, and the biological observation kettle and performing gas pressurization on the sample mixing kettle, the sample settling kettle, the sample purification kettle, the enrichment kettle, and the biological observation kettle; and installing each unit of the online purification and sampling and visualized automatic enrichment apparatus for deep-sea microorganisms;
S2: acquiring deep-sea microorganism samples from the sample container and transferring the samples to the sample mixing kettle, activating the pressure-retaining stirrer inside the sample mixing kettle, thoroughly mixing the samples after a first preset duration, and obtaining the samples mixed;
S3: transferring the samples mixed to the sample settling kettle, using the turbidimeter to monitor sedimentation and separation of impurities of the samples in real time, and obtaining samples settled when turbidity drops to a first preset threshold;
S4: transferring the samples settled to the sample purification kettle, activating the pressure-retaining stirrer inside the sample purification kettle, driving the purification piston for purification, and using the total organic carbon analyzer to monitor the purification process of the samples in real time; when total organic carbon content obtained by the total organic carbon analyzer exceeds a second preset threshold, obtaining the samples purified and transferring the samples purified to the enrichment kettle;
S5: activating the magnetic stirrer to initiate enrichment, and using the environmental parameter sensing system to monitor environmental indicators inside the enrichment kettle in real time; when monitored environmental indicators meet preset conditions, obtaining an enriched microbial liquid and completing enrichment;
S6: opening the measurement solenoid valve to transfer the enriched microbial liquid to the biological abundance measurement system inside the biological observation kettle, using the microscopic imaging system for biological abundance observation, and after completing observation, opening the recovery solenoid valve to transfer the enriched microbial liquid observed back to the enrichment kettle for recovery; and
S7: repeating steps S5 and S6, performing multiple rounds of enrichment and observation until observed biological abundance reaches a third preset threshold, and collecting all enriched microbial liquid from the enrichment kettle for further preservation or experimentation.
